(19) United States
(12) Patent Application Publication
(10) Pub. No.: US 2017/0151164 A1
(43) Pub. Date: Jun. 1, 2017

(54) HIGH-PERFORMANCE HAIR TREATMENT AGENTS WITH AN ANTI-WASHOUT EFFECT

(71) Applicant: Henkel AG & Co. KGaA, Duesseldorf (DE)

(72) Inventors: Volker Scheunemann, Lueneburg (DE); Erik Schulze zur Wiesche, Hamburg (DE)

(73) Assignee: Henkel AG & Co. KGaA, Duesseldorf (DE)

(21) Appl. No.: 15/364,985

(22) Filed: Nov. 30, 2016

(30) Foreign Application Priority Data
Dec. 1, 2015 (DE) 10 2015 223 855.5

(51) Int. Cl. A61K 8/898; A61K 8/42; A61K 8/73; A61K 8/365; A61Q 5/00; A61K 8/362; A61K 8/33; A61Q 5/02; A61Q 5/12; A61K 8/46; A61K 8/36

(52) U.S. Cl. CPC A61K 8/898; A61K 8/463; A61K 8/42; A61K 8/731; A61K 8/365; A61K 8/36; A61K 8/362; A61K 8/33; A61Q 5/02; A61Q 5/12; A61Q 5/004; A61K 2800/596

(57) ABSTRACT

Hair treatment agents containing at least one anionic surfactant, at least one amphoteric and/or nonionic surfactant, at least one cellulose-based cationic polymer, at least one amino-functional silicone, at least one organic acid, and phenoxyisopropanol reduce or prevent the washing out of color from dyed hair.

### CROSS-REFERENCE TO RELATED APPLICATION

This application claims priority to German Patent Application No. 10 2015 223 855.5, filed Dec. 1, 2015, which is incorporated herein by reference in its entirety.

### TECHNICAL FIELD

The disclosure relates to hair treatment agents, in particular shampoos and so-called conditioners, with an active substance combination for the gentle and effective care of hair.

### BACKGROUND

The importance of care products with the longest-lasting possible effect is increasing not least due to the great stressing of hair, for example, by coloring or permanents, as well as by cleaning hair with shampoos and by harmful environmental factors. Care products of this type influence the natural structure and properties of hair. Thus, after such care treatments, for example, the wet and dry combability of hair, the hold, and fullness of the hair can be optimized or the hair can be protected from an increase in split ends.

It has long been customary, therefore, to subject hair to a special aftertreatment. In this regard, the hair is treated with special active substances, for example, quaternary ammonium salts or special polymers, usually in the form of a rinse. Depending on the formulation, the combability, hold, and fullness of the hair can be improved by this treatment and the splitting rate reduced.

Multifunctional cosmetic products are also known in the prior art. These include in particular the so-called “2 in 1” shampoos, which not only clean the hair but also condition it. Such products are highly valued by the consumer, because due to their product performance they make at least one process step unnecessary, for example, the conditioning with a classic hair rinse.

Similarly, products for modifying the natural color of hair play a prominent role in hair cosmetics. Permanent, semi-permanent, or temporary coloring systems are differentiated, which systems are based on chemical and/or natural dyes. The hair colors produced artificially by permanent, semi-permanent, or temporary coloring systems have the disadvantage, however, that they can change in an undesirable way, for example, during or after the cleaning of hair.

An “undesirable change” is understood to be the fading or bleeding and the loss of color brilliance of the hair shade achieved by the particular dyeing. Environmental effects and/or the effects of sunlight can intensify these changes still further.

The use of divalent metal salts in hair dyes to improve the durability and thereby the fastness of the color is known from EP 2438900 A1.

There continues to be a need to provide active substances or active substance combinations for hair treatment agents with good care properties, which, moreover, strengthen the adhesion of dyes to hair fibers and thus maintain the fastness of the artificially produced hair color, and in this respect to develop hair treatment agents further.

It was observed, however, that water hardness at times can have a negative effect on the care properties of hair treatment agents, so that the care performance of one the same product is either too low or too high depending on the water quality.

A too high care performance or “overcare” (occurrence of a so-called build-up effect) of the hair is understood primarily as a greasy hair feel, smaller hair volume, and/or a stringy, unkempt appearance of the hair, whereas damaged (brittle, prone to splitting, dull) hair is an indication of a too low care performance.

### BRIEF SUMMARY

Hair treatment agents and methods are provided herein. In an embodiment, a hair treatment agent includes:
a) at least one anionic surfactant,
b) at least one amphoteric and/or nonionic surfactant,
c) at least one cellulose-based cationic polymer,
d) at least one amino-functional silicone,
e) at least one organic acid, and
f) phenoxyisopropanol

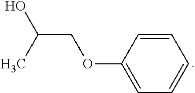

In another embodiment, a method for treating hair includes applying a hair treatment agent to dry or damp hair, wherein the hair treatment agent includes:
a) at least one organic acid, at least one anionic surfactant,
b) at least one amphoteric and/or nonionic surfactant,
c) at least one cellulose-based cationic polymer,
d) at least one amino-functional silicone,
e) at least one organic acid, and
f) phenoxyisopropanol

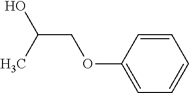

where the hair treatment is agent is left on the hair for a time period of about 30 to about 300 seconds, and is rinsed out thereafter.

### DETAILED DESCRIPTION

The following detailed description is merely exemplary in nature and is not intended to limit the hair treatment agents and methods for treating hair. Furthermore, there is no intention to be bound by any theory presented in the preceding background or the following detailed description.

The object of the present application therefore was to provide highly skin-compatible hair treatment agents with care properties, which have excellent foam properties and have a good rinsability with water and, moreover, reduce or prevent the washing out of color from dyed hair.

The hair treatment agents with care properties should have, independent of the water quality, an unvarying conditioning performance and not weigh down in particular fine hair and/or damaged hair and thereby strengthen the hair's structure and thus protect from splitting and breaking and improve combability and feel. Moreover, it should have a germ-reducing effect.

It has now been found that a combination of certain ingredients has an especially positive effect on dyed hair treated therewith and on the hair follicle.

A first subject as contemplated herein is hair treatment agents, containing
a) at least one anionic surfactant,
b) at least one amphoteric and/or nonionic surfactant,
c) at least one cellulose-based cationic polymer,
d) at least one amino-functional silicone,
e) at least one organic acid, and
f) phenoxyisopropanol

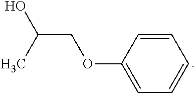

Hair treatment agents in the context as contemplated herein are, for example, hair shampoos, hair conditioners, conditioning shampoos, hair sprays, hair rinses, hair treatments, hair packs, hair tonics, permanent wave fixing solutions, hair coloring shampoos, hair dyes, hair setting lotions, hair setting products, hair styling preparations, blow-dry wave lotions, styling mousses, hair gels, hair waxes, or combinations thereof. In view of the fact that men in particular are often reluctant to use multiple different agents and/or multiple application steps, such agents are preferred that a man uses in any event. Preferred agents therefore are shampoos, conditioning agents, or hair tonics.

The hair treatment agents contain at least one anionic surfactant.

All anionic surface-active substances, suitable for use on the human body, are suitable as anionic surfactants and emulsifiers for the compositions contemplated herein. These are characterized by an anionic group imparting water solubility, such as, e.g., a carboxylate, sulfate, sulfonate, or phosphate group, and a lipophilic alkyl group having about 8 to 30 C atoms. In addition, the molecule can contain glycol ether or polyglycol ether groups, ester, ether, and amide groups, and hydroxyl groups. Examples of suitable anionic surfactants and emulsifiers are, each in the form of the sodium, potassium, and ammonium and mono-, di-, and trialkanolammonium salts having 2 to 4 C atoms in the alkanol group,
linear and branched fatty acids having 8 to 30 C atoms (soaps),
ether carboxylic acids of the formula R—O—(CH₂—CH₂O)ₓ—CH₂—COOH, in which R is a linear alkyl group having 8 to 30 C atoms and x=0 or is 1 to 16,
acyl sarcosides having 8 to 24 C atoms in the acyl group,
acyl taurides having 8 to 24 C atoms in the acyl group,
acyl isethionates having 8 to 24 C atoms in the acyl group,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms,
acyl glutamates of the formula (T-I),

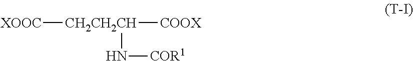

in which R¹CO stands for a linear or branched acyl group having 6 to 22 carbon atoms and 0, 1, 2, or 3 double bonds and X for hydrogen, an alkali and/or alkaline earth metal, ammonium, alkyl ammonium, alkanol ammonium, or glucammonium, for example, acyl glutamates, derived from fatty acids having 6 to 22, preferably 12 to 18 carbon atoms, such as, for example, C_{12/14} or C_{12/18} coconut fatty acid, lauric acid, myristic acid, palmitic acid, and/or stearic acid, in particular sodium N-cocoyl- and sodium N-stearoyl-L-glutamate,
esters of a hydroxy-substituted di- or tricarboxylic acid of the general formula (T-II),

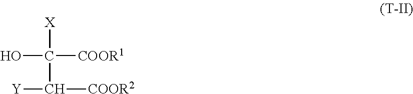

in which X=H or is a —CH₂COOR group, Y=H or is —OH on condition that Y=H, if X=—CH₂COOR, R, R¹, and R² independently of one another denote a hydrogen atom, an alkali or alkaline earth metal cation, an ammonium group, the cation of an ammonium-organic base, or a Z group, which originates from a polyhydroxylated organic compound, selected from the group of etherified (C₆-C₁₈) alkyl polysaccharides with 1 to 6 monomeric saccharide units and/or etherified aliphatic (C₆-C₁₆) hydroxyalkylpolyols with 2 to 16 hydroxyl groups, with the proviso that at least one of the R, R¹, or R² groups is a Z group,
esters of sulfosuccinic acid or sulfosuccinates of the general formula (T-III),

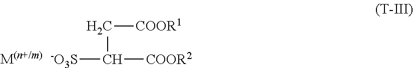

in which M^{(n+/n)} for n=1 represents a hydrogen atom, an alkali metal cation, an ammonium group, or the cation of an ammonium-organic base and for n=2 an alkaline earth metal cation, and R¹ and R² independently of one another denote a hydrogen atom, an alkali or alkaline earth metal cation, an ammonium group, the cation of an ammonium-organic base, or a Z group, which originates from a polyhydroxylated organic compound, selected from the group of etherified (C₆-C₁₈) alkyl polysaccharides with 1 to 6 monomeric saccharide units and/or etherified aliphatic (C₆-C₁₆) hydroxyalkylpolyols with 2 to 16 hydroxyl groups, with the proviso that at least one of the R¹ or R² group is a Z group,
sulfosuccinic acid mono- and dialkyl esters having 8 to 24 C atoms in the alkyl group and sulfosuccinic acid monoalkylpolyoxyethyl esters having 8 to 24 C atoms in the alkyl group and 1 to 6 oxyethyl groups,
alkyl sulfates and alkyl polyglycol ether sulfates of the formula R—(O—CH₂—CH₂)ₓ—OSO₃H, in which R is a preferably linear alkyl group having 8 to 30 C atoms and x=0 or is 1 to 12,
mixed surface-active hydroxysulfonates according to DE-A-37 25 030,
esters of tartaric acid and citric acid with alcohols, representing adducts of about 2 to 15 molecules of ethylene oxide and/or propylene oxide to C₈-C₂₂ fatty alcohols,
alkyl and/or alkenyl ether phosphates,
sulfated fatty acid alkylene glycol esters, and
monoglyceride sulfates and monoglyceride ether sulfates.

Preferred anionic surfactants and emulsifiers are acyl glutamates, acyl isethionates, acyl sarcosinates, and acyl taurates, in each case with a linear or branched acyl group having 6 to 22 carbon atoms and 0, 1, 2, or 3 double bonds, which in particularly preferred embodiments are selected from an octanoyl, decanoyl, lauroyl, myristoyl, palmitoyl, and stearoyl group, esters of tartaric acid, citric acid, or succinic acid or the salts of said acids with alkylated glucose, in particular the products with the INCI name Disodium Coco-Glucoside Citrate, Sodium Coco-Glucoside Tartrate, and Disodium Coco-Glucoside Sulfosuccinate, alkyl polyglycol ether sulfates and ether carboxylic acids having 8 to 18 C atoms in the alkyl group and up to 12 ethoxy groups in the molecule, sulfosuccinic acid mono- and dialkyl esters having 8 to 18 C atoms in the alkyl group, and sulfosuccinic acid monoalkylpolyoxyethyl esters having 8 to 18 C atoms in the alkyl group and 1 to 6 ethoxy groups.

Further preferred anionic surfactants are alkyl sulfates, alkyl polyglycol ether sulfates, and ether carboxylic acid salts having 10 to 18 C atoms in the alkyl group and up to 12 glycol ether groups in the molecule, and sulfosuccinic acid mono- and dialkyl esters having 8 to 18 C atoms in the alkyl group, and sulfosuccinic acid monoalkyl polyoxyethyl esters having 8 to 18 C atoms in the alkyl group and 1 to 6 oxyethyl groups.

Particularly preferred anionic surfactants are the alkali or ammonium salts of lauryl ether sulfate with an ethoxylation degree of 2 to 4 EO.

Preferred hair treatment agents contain, based on their weight, 0.5 to 20% by weight, preferably 0.75 to 15% by weight, more preferably 1 to 12% by weight, and in particular 2 to 10% by weight of anionic surfactant(s).

Particularly preferred hair treatment agents contemplated herein are characterized in that, based on their weight, they contain 0.5 to 20% by weight, preferably 0.75 to 15% by weight, more preferably 1 to 12% by weight, and in particular 2 to 10% by weight of alkyl (ether) sulfates of the general formula R—(OCH₂—CH₂)ₙ—OSO₃X, in which R denotes a straight-chain or branched, saturated or unsaturated alkyl group having 8 to 24 C atoms, n the number 0 or 1 to 12, and X an alkali, alkaline earth, ammonium, or alkanolamine ion.

The hair treatment agents contemplated herein contain at least one amphoteric surfactant and/or at least one nonionic surfactant. Surfactants that have both a negatively and also a positively charged functional group are called amphoteric surfactants or zwitterionic surfactants as well.

Particularly suitable zwitterionic surfactants are the so-called betaines, such as the N-alkyl-N,N-dimethylammonium glycinates, for example, coco alkyl dimethylammonium glycinate and N-acylaminopropyl-N,N-dimethylammonium glycinates, for example, cocoacylaminopropyl dimethylammonium glycinate, and 2-alkyl-3-carboxymethyl-3-hydroxyethyl imidazolines each having 8 to 18 C atoms in the alkyl or acyl group, as well as coco acylaminoethyl hydroxyethyl carboxymethyl glycinate. A preferred zwitterionic surfactant is the fatty acid amide derivative known under the INCI name of Cocamidopropyl Betaine.

Further examples of suitable ampholytic surfactants are N-alkylglycines, N-alkylpropionic acids, N-alkylaminobutyric acids, N-alkyliminodipropionic acids, N-hydroxyethyl-N-alkylamidopropyl glycines, N-alkyltaurines, N-alkylsarcosines, 2-alkylaminopropionic acids, and alkylaminoacetic acids each having about 8 to 24 C atoms in the alkyl group.

Particularly preferred ampholytic surfactants are N-cocoalkylaminopropionate, cocoacylaminoethylaminopropionate, and C₁₂-C₁₈ acylsarcosine.

Preferred hair treatment agents contemplated herein contain, based on their weight, about 0.3 to about 10% by weight, preferably about 0.5 to about 8% by weight, more preferably about 0.75 to about 6% by weight, and in particular about 1 to 5% about by weight of amphoteric surfactant(s).

Particularly preferred hair treatment agents contemplated herein are characterized in that they contain amphoteric surfactant(s) from the groups of
N-alkylglycines,
N-alkylpropionic acids,
N-alkylaminobutyric acids,
N-alkyliminodipropionic acids,
N-hydroxyethyl-N-alkylamidopropylglycines,
N-alkyltaurines,
N-alkylsarcosines,
2-alkylaminopropionic acids each having about 8 to 24 C atoms in the alkyl group,
alkylaminoacetic acids each having about 8 to 24 C atoms in the alkyl group,
N-cocoalkylaminopropionate,
cocoacylaminoethylaminopropionate,
C₁₂-C₁₈ acylsarcosine,
N-alkyl-N,N-dimethylammonium glycinates, for example, cocoalkyldimethylammonium glycinate,
N-acylaminopropyl-N,N-dimethylammonium glycinates, for example, cocoacylaminopropyldimethylammonium glycinate,
2-alkyl-3-carboxymethyl-3-hydroxyethyl imidazolines each having 8 to 18 C atoms in the alkyl or acyl group,
cocoacylaminoethylhydroxyethylcarboxymethyl glycinate,
the compounds known under the INCI name Cocamidopropyl Betaine,
the compounds known under the INCI name Disodium Cocoamphodiacetate,
   preferred agents containing the amphoteric surfactant(s) in amounts of about 0.3 to about 10% by weight, preferably about 0.5 to about 8% by weight, more preferably about 0.75 to about 6% by weight, and in particular about 1 to about 5% by weight, based in each case on the total agent.

Particularly preferred hair treatment agents contain as amphoteric surfactants betaines of the formula (Bet-I)

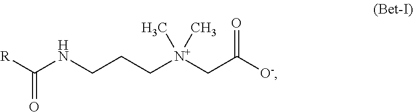

in which R stands for a straight-chain or branched, saturated or mono- or polyunsaturated alkyl or alkenyl group having 8 to 24 carbon atoms.

These surfactants are called Amidopropylbetaines according to the INCI nomenclature, the representatives, derived from coconut fatty acids, being preferred and being called cocamidopropyl betaines. Used with particular preference as contemplated herein are surfactants of the formula (Bet-I), which are a mixture of the following representatives:

H₃C—(CH₂)₇—C(O)—NH—(CH₂)₃N⁺(CH₃)₂CH₂COO⁻
H₃C—(CH₂)₉—C(O)—NH—(CH₂)₃N⁺(CH₃)₂CH₂COO⁻
H₃C—(CH₂)₁₁—C(O)—NH—(CH₂)₃N⁺(CH₃)₂CH₂COO⁻
H₃C—(CH₂)₁₃—C(O)—NH—(CH₂)₃N⁺(CH₃)₂CH₂COO⁻
H₃C—(CH₂)₁₅—C(O)—NH—(CH₂)₃N⁺(CH₃)₂CH₂COO⁻
H₃C—(CH₂)₇—CH═CH—(CH₂)₇—C(O)—NH—(CH₂)₃N⁺(CH₃)₂CH₂COO⁻

Used with particular preference are surfactants of the formula (Bet-I) within rather narrow amount ranges. Here hair treatment agents contemplated herein are preferred, which contain, based on their weight, about 0.25 to about 8% by weight, more preferably about 0.5 to about 7% by weight, more preferably about 0.75 to about 6.5% by weight, and in particular about 1 to about 5.5% by weight of surfactant(s) of the formula (Bet-I).

In addition to amphoteric surfactant(s) of the formula (Bet-I) or in their place, the hair treatment agents contemplated herein can contain with particular preference as amphoteric surfactants betaines of the formula (Bet-II)

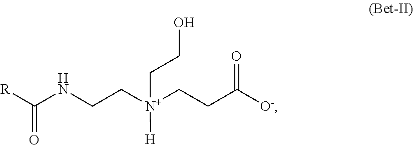

in which R stands for a straight-chain or branched, saturated or mono- or polyunsaturated alkyl or alkenyl group having 8 to 24 carbon atoms.

These surfactants are called Amphoacetates according to the INCI nomenclature, the representatives, deriving from coconut fatty acids, being preferred and being called Cocoamphoacetates.

For production-related reasons, surfactants of this type always also contain betaines of the formula (Bet-IIa)

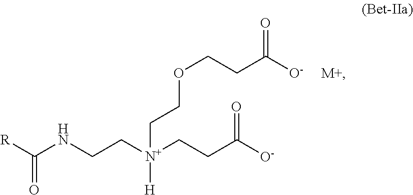

in which R stands for a straight-chain or branched, saturated or mono- or polyunsaturated alkyl or alkenyl group having 8 to 24 carbon atoms and M for a cation.

These surfactants are called Amphodiacetates according to the INCI nomenclature, the representatives, deriving from coconut fatty acids, being preferred and being called Cocoamphodiacetates.

Particularly preferred as contemplated herein are surfactants of the formula (Bet-II), which are a mixture of the following representatives:

H₃C—(CH₂)₇—C(O)—NH—(CH₂)₂NH⁺(CH₂CH₂OH)CH₂CH₂COO⁻
H₃C—(CH₂)₉—C(O)—NH—(CH₂)₂NH⁺(CH₂CH₂OH)CH₂CH₂COO⁻
H₃C—(CH₂)₁₁—C(O)—NH—(CH₂)₂NH⁺(CH₂CH₂OH)CH₂CH₂COO⁻
H₃C—(CH₂)₁₃—C(O)—NH—(CH₂)₂NH⁺(CH₂CH₂OH)CH₂CH₂COO⁻
H₃C—(CH₂)₁₅—C(O)—NH—(CH₂)₂NH⁺(CH₂CH₂OH)CH₂CH₂COO⁻
H₃C—(CH₂)₇—CH═CH—(CH₂)₇—C(O)—NH—(CH₂)₂NH⁺(CH₂CH₂OH)CH₂CH₂COO⁻

Used with particular preference are surfactants of the formula (Bet-II) within rather narrow amount ranges. Here hair treatment agents contemplated herein are preferred, which contain, based on their weight, about 0.25 to about 8% by weight, more preferably about 0.5 to about 7% by weight, more preferably about 0.75 to about 6.5% by weight, and in particular about 1 to about 5.5% by weight of surfactant(s) of the formula (Bet-II).

In summary, cosmetic agents contemplated herein are preferred in which the R group in the formulas (Bet-I) and (Bet-II) is selected from

H₃C—(CH₂)₇— H₃C—(CH₂)₉— H₃C—(CH₂)₁₁— H₃C—(CH₂)₁₃— H₃C—(CH₂)₁₅— H₃C—(CH₂)₇—CH═CH—(CH₂)₇—, or mixtures thereof.

The hair treatment agents can contain nonionic surfactant(s). If they are free of amphoteric surfactants, they must contain a nonionic surfactant.

Suitable nonionic surfactants include, for example,
adducts of 4 to 30 mol of ethylene oxide and/or 0 to 5 mol of propylene oxide to linear fatty alcohols having 8 to 22 C atoms, to fatty acids having 12 to 22 C atoms and to alkyl phenols having 8 to 15 C atoms in the alkyl group,
ethylene oxide and polyglycerol adducts to methylglucoside fatty acid esters, fatty acid alkanolamides, and fatty acid glucamides,
C₈-C₃₀ fatty acid monoesters and diesters of adducts of 1 to 30 mol of ethylene oxide to glycerol,
amine oxides,
sorbitan fatty acid esters and adducts of ethylene oxide to sorbitan fatty acid esters such as, for example, polysorbates,
fatty acid alkanolamides of the following general formula,

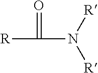

in which R preferably denotes a linear or branched, saturated or unsaturated alkyl or alkenyl group having 8 to 24 carbon atoms and the R′ groups stand for hydrogen or for the group —(CH₂)ₙOH, in which n denotes the numbers 2 or 3, with the proviso that at least one of the R′ groups stands for the aforementioned group —(CH₂)ₙOH,
sugar fatty acid esters and adducts of ethylene oxide to sugar fatty acid esters,
adducts of ethylene oxide to fatty acid alkanolamides and fatty amines, and/or
alkyl (oligo)glucosides,
mixtures of alkyl (oligo)glucosides and fatty alcohols, for example, the commercially available product Montanov® 68,
adducts of about 5 to about 60 mol of ethylene oxide to castor oil and hydrogenated castor oil,
partial esters of polyols having 3 to 6 carbon atoms with saturated fatty acids having 8 to 22 C atoms,
sterols. Sterols are understood to be a group of steroids that have a hydroxyl group at C atom 3 of the steroid skeleton, and are isolated from both animal tissue (zoosterols) and vegetable fats (phytosterols). Examples of zoosterols are cholesterol and lanosterol. Examples of suitable phytosterols are ergosterol, stigmasterol, and sitosterol. Sterols, the so-called mycosterols, are also isolated from fungi and yeasts.
phospholipids. These are primarily glucose phospholipids, which are obtained, e.g., as lecithins or phosphatidylcholines from, e.g., egg yolk or plant seeds (e.g., soybeans).

Suitable alkyl (oligo)glycosides can be selected from compounds of the general formula RO-[G]ₓ, in which [G] is derived preferably from aldoses and/or ketoses having 5-6 carbon atoms, preferably from glucose.

The subscript x stands for the degree of oligomerization (DP), i.e., for the distribution of the mono- and oligoglycosides. The subscript x preferably has a value in the range of 1 to 10, particularly preferably in the range of 1 to 3; this value cannot be an integer but can be a fraction, which can be determined analytically.

Particularly preferred alkyl (oligo)glycosides have a degree of oligomerization between 1.2 and 1.5.

The R group preferably stands for at least one alkyl and/or alkenyl group having 4 to 24 C atoms.

Particularly preferred alkyl (oligo)glycosides are the compounds known under the INCI names Caprylyl/Capryl Glucoside, Decyl Glucoside, Lauryl Glucoside, and Coco Glucoside.

Suitable amine oxides can be selected from at least one compound of the general formula (A-I) or (A-II)

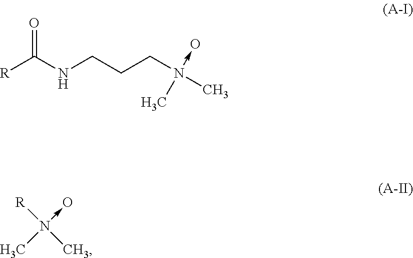

in which R in each case stands for a straight-chain or branched, saturated or mono- or polyunsaturated alkyl or alkenyl group having 6 to 24 carbon atoms, preferably having 8 to 18 carbon atoms.

The surfactants of the aforementioned formula (A-I) or (A-III), obtainable commercially from various suppliers and known under the INCI names Cocamine Oxide, Lauramine Oxide, and/or Cocamidopropyl Amine Oxide are preferred in particular.

Of the suitable C₈-C₃₀ fatty acid mono- and diesters of adducts of about 1 to about 30 mol of ethylene oxide to glycerol, understood as preferred are those known under the INCI names PEG(1-10) Glyceryl Cocoate, in particular PEG-7 Glyceryl Cocoate.

It can be advantageous, furthermore, to combine the ethoxylated fatty acid esters with other ethoxylated fatty acid esters. Such product mixtures are commercially available, for example, under the name “Antil 200®” (INCI name: PEG-200 Hydrogenated Glyceryl Palmate, PEG-7 Glyceryl Cocoate) from the company Evonik.

Particularly preferred nonionic surfactants, which may be present in the hair treatment agents contemplated herein, are fatty acid alkanolamides, in particular the compounds known under the INCI names Cocamide MEA and/or Cocamide MIPA; alkyl (oligo)glucosides, in particular the compounds known under the INCI names Caprylyl/Capryl Glucoside, Decyl Glucoside, Lauryl Glucoside, and/or Coco Glucoside; C₈-C₃₀ fatty acid monoesters and diesters of adducts of about 1 to about 30 mol of ethylene oxide to glycerol, in particular the compound known under the INCI name PEG-7 Glyceryl Cocoate; and/or adducts of about 4 to about 30 mol of ethylene oxide and/or 0 to about 5 mol of propylene oxide to linear fatty alcohols having 8 to 22 C atoms.

Cocamide MEA and/or PEG-7 Glyceryl Cocoate are particularly preferred because of their foam-stabilizing and refatting properties.

Hair treatment agents preferred as contemplated herein contain, based on their weight, about 0.3 to about 10% by weight, preferably about 0.5 to about 8% by weight, more preferably about 0.75 to about 6% by weight, and in particular about 1 to about 5% by weight of nonionic surfactant(s).

The hair treatment agents can contain cationic surfactant(s). Cationic surfactants of the type of quaternary ammonium compounds, esterquats, and amidoamines are usable as contemplated herein. Preferred quaternary ammonium compounds are ammonium halides, particularly chlorides and bromides, such as alkyltrimethylammonium chlorides, dialkyldimethylammonium chlorides, and trialkylmethylammonium chlorides. The long alkyl chains of these surfactants preferably have 10 to 18 carbon atoms, such as, e.g., cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride. Further preferred cationic surfactants are the imidazolium compounds known under the INCI names Quaternium-27 and Quaternium-83.

Particularly preferred hair treatment agents contemplated herein are characterized in that they contain as a cationic care substance, based on their weight, about 0.05 to about 7.5% by weight, preferably about 0.1 to about 5% by weight, particularly preferably about 0.2 to about 3.5% by weight, and in particular about 0.25 to about 2.5% by weight of cationic surfactant(s) from the group of quaternary ammonium compounds and/or esterquats and/or amidoamines, preferred cationic surfactant(s) being selected from
alkyltrimethylammonium chlorides having preferably 10 to 18 carbon atoms in the alkyl group and/or
dialkyldimethylammonium chlorides having preferably 10 to 18 carbon atoms in the alkyl group and/or
trialkylmethylammonium chlorides having preferably 10 to 18 carbon atoms in the alkyl group and/or
cetyltrimethylammonium chloride and/or
stearyltrimethylammonium chloride and/or
distearyldimethylammonium chloride and/or
lauryldimethylammonium chloride and/or
lauryldimethylbenzylammonium chloride and/or
tricetylmethylammonium chloride
Quaternium-27 and/or
Quaternium-83 and/or
N-methyl-N(2-hydroxyethyl)-N,N-(ditalgacyloxyethyl)ammonium methosulfate and/or
N-methyl-N(2-hydroxyethyl)-N,N-(distearoyloxyethyl)ammonium methosulfate and/or
N,N-dimethyl-N,N-distearoyloxyethylammonium chloride and/or
N,N-di-(2-hydroxyethyl)-N,N-(fatty acid ester ethyl)ammonium chloride.

The hair treatment agents contain at least one cellulose-based cationic polymer.

Cellulose-based cationic polymers increase the care performance of the hair treatment agents contemplated herein (in particular the effectiveness of the agents contemplated herein against hair breakage). Suitable cellulose-based cationic polymers are also called cationic cellulose compounds.

Particularly preferred hair treatment agents contemplated herein contain as cellulose-based cationic polymer(s) c), based on the weight of the agent, about 0.01 to about 3% by weight, preferably about 0.05 to about 2% by weight, more preferably about 0.1 to about 1.5% by weight, and in particular about 0.15 to about 0.8% by weight of at least one polymer from the group of cationic cellulose polymers.

Cationic cellulose compounds in the context contemplated herein are those that bear more than one permanent cationic charge in at least one side chain. Cellulose is made up of beta-1,4-glycosidically linked D-glucopyranose units and forms unbranched, water-insoluble chains. Chemical substituents that bond to the cellulose backbone and are not found in native cellulose, because they are introduced subsequently, e.g., by chemical synthesis, are defined as a “side chain” of a cellulose.

Preferred are quaternized cellulose polymers that result from hydroxy(C₂-C₄)alkyl celluloses, particularly preferably from hydroxyethyl celluloses.

Such polymers are known to the skilled artisan and available commercially from various companies. Particularly preferred are the cationic cellulose derivatives known under the INCI names Polyquaternium-4, Polyquaternium-10, Polyquaternium-24, Polyquaternium-67, and/or Polyquaternium-72. Very particularly preferred are Polyquaternium-10, Polyquaternium-24, and/or Polyquaternium-67, and Polyquaternium-10 is preferred in particular.

Preferred hair treatment agents contemplated herein contain as cationic polysaccharide polymer(s) c), based on the weight of the agent, about 0.01 to about 3% by weight, preferably about 0.05 to about 2% by weight, more preferably about 0.1 to about 1.5% by weight, and in particular about 0.15 to about 0.8% by weight of at least one polymer from the group of Polyquaternium-4, Polyquaternium-10, Polyquaternium-24, Polyquaternium-67, and/or Polyquaternium-72.

Particularly preferred hair treatment agents contemplated herein contain as cationic polysaccharide polymer(s) c), based on the weight of the agent, about 0.01 to about 3% by weight, preferably about 0.05 to about 2% by weight, more preferably about 0.1 to about 1.5% by weight, and in particular about 0.15 to about 0.8% by weight of Polyquaternium-10.

The hair treatment agents contain at least one amino-functional silicone. Such silicones can be described, e.g., by the formula

M(RₐQ_{b}SiO_{(4-a-b)/2})ₓ(R_{c}SiO_{(4-c)/2})_{y}M,

wherein R in the above formula is a hydrocarbon or a hydrocarbon group having 1 to about 6 carbon atoms, Q is a polar group of the general formula —R¹HZ, wherein R¹ is a divalent linking group that is bound to hydrogen and the Z group and is made up of carbon and hydrogen atoms, carbon, hydrogen, and oxygen atoms or carbon, hydrogen, and nitrogen atoms, and Z is an organic, amino-functional group that contains at least one amino-functional group; “a” assumes values in the range of about 0 to about 2, “b” assumes values in the range of about 1 to about 3, “a”+“b” is less than or equal to 3, and “c” is a number in the range of about 1 to about 3, and x is a number in the range of 1 to about 2000, preferably of about 3 to about 50, and most preferably of about 3 to about 25, and y is a number in the range of about 20 to about 10,000, preferably of about 125 to about 10,000, and most preferably of about 150 to about 1000, and M is a suitable silicone end group, as is known in the state of the art, preferably trimethylsiloxy. Non-limiting examples of the groups represented by R include alkyl groups, such as methyl, ethyl, propyl, isopropyl, isopropyl, butyl, isobutyl, amyl, isoamyl, hexyl, isohexyl, and the like; alkenyl groups such as vinyl, halovinyl, alkylvinyl, allyl, haloallyl, and alkylallyl; cycloalkyl groups such as cyclobutyl, cyclopentyl, cyclohexyl, and the like; phenyl groups, benzyl groups, halohydrocarbon groups such as 3-chloropropyl, 4-bromobutyl, 3,3,3-trifluoropropyl, chlorocyclohexyl, bromophenyl, chlorophenyl, and the like, and sulfur-containing groups such as mercaptoethyl, mercaptopropyl, mercaptohexyl, mercaptophenyl, and the like; R is preferably an alkyl group, containing 1 to about 6 carbon atoms, and most preferably R is methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—, and —(CH₂)₃C(O)SCH₂CH₂—.

Z is an organic, amino-functional group containing at least one functional amino group. A possible formula for Z is NH(CH₂)_{z}NH₂, where z is 1 or more. Another possible formula for Z is —NH(CH₂)_{z}(CH₂)_{zz}NH, where both z and zz are independently 1 or more, this structure comprising diamino ring structures such as piperazinyl. Z is most preferably a —NHCH₂CH₂NH₂ group. Another possible formula for Z is —N(CH₂)_{z}(CH₂)_{zz}NX₂ or —NX₂, where each X of X₂ is selected independently from the group consisting of hydrogen and alkyl groups having 1 to 12 carbon atoms, and zz is 0.

Q is most preferably a polar, amino-functional group of the formula —CH₂CH₂CH₂NHCH₂CH₂NH₂. In the formulas, “a” assumes values in the range of about 0 to about 2, “b” assumes values in the range of about 2 to about 3, “a”+“b” is less than or equal to 3, and “c” is a number in the range of about 1 to about 3. The molar ratio of the RₐQ_{b} SiO_{(4-a-b)/2} units to the R_{c}SiO_{(4-c)/2} units is in the range of about 1:2 to 1:65, preferably from about 1:5 to about 1:65, and most preferably from about 1:15 to about 1:20. If one or more silicones of the above formula are used, then the various variable substituents in the above formula can be different in the various silicone components, present in the silicone mixture.

Preferred agents contemplated herein are characterized in that they contain an amino-functional silicone of the formula (Si-II)

R′ₐG₃₋ₐ-Si(OSiG₂)ₙ—(OSiG_{b}R′_{2-b})ₘ—O-SiG₃₋ₐ-R′ₐ (Si-II),

in which:
G is —H, a phenyl group, OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a stands for a number between 0 and 3, in particular 0;
b stands for a number between 0 and 1, in particular 1,
m and n are numbers whose sum (m+n) amounts to between 1 and 2000, preferably between 50 and 150, n preferably assuming values from 0 to 1999 and in particular from 49 to 149 and m preferably assuming values from 1 to 2000, in particular from 1 to 10,
R′ is a monovalent group selected from
   -Q-N(R″)—CH₂—CH₂—N(R″)₂
   -Q-N(R″)₂
   -Q-N⁺(R″)₃A⁻
   -Q-N⁺H₂(R″)₂A⁻
   -Q-N⁺H₂(R″)A⁻
   -Q-N(R″)—CH₂—CH₂—N⁺R″H₂A⁻,
wherein each Q stands for a chemical bond, —CH₂—, —CH₂—CH₂—, —CH₂CH₂CH₂—, —C(CH₃)₂, —CH₂CH₂CH₂CH₂—, —CH₂C(CH₃)₂—, —CH(CH₃)CH₂CH₂—,
R″ stands for identical or different groups from the group —H, -phenyl, -benzyl, CH₂—CH(CH₃)Ph, C₁₋₂₀ alkyl groups, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃, and A represents an anion, which is preferably selected from chloride, bromide, iodide, or methosulfate.

Particularly preferred agents contemplated herein are characterized in that they contain at least one amino-functional silicone of the formula (Si-IIa)

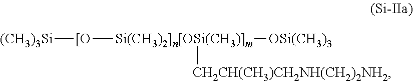

in which m and n are numbers whose sum (m+n) amounts to between 1 and 2000, preferably between 50 and 150, n preferably assuming values from 0 to 1999 and in particular from 49 to 149 and m preferably assuming values from 1 to 2000, in particular from 1 to 10.

These silicones are designated as Trimethylsilylamodimethicone in accordance with the INCI Declaration.

Particularly preferred as well are agents contemplated herein that contain an amino-functional silicone of the formula (Si-IIb)

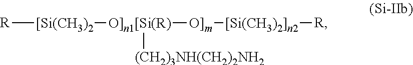

in which R stands for —OH, —O—CH₃, or a —CH₃ group and m, n1 and n2 are numbers, whose sum (m+n1+n2) amounts to between 1 and 2000, preferably between 50 and 150, the sum (n1+n2) preferably assuming values from 0 to 1999 and in particular from 49 to 149 and m preferably assuming values from 1 to 2000, in particular from 1 to 10.

These silicones are designated as Amodimethicone in accordance with the INCI Declaration.

Irrespective of which amino-functional silicones are used, agents contemplated herein are preferred that contain an amino-functional silicone whose amine value is above 0.25 mEq/g, preferably above 0.3 mEq/g, and in particular above 0.4 mEq/g. The amine value here stands for the milliequivalents of amine per gram of the amino-functional silicone. It can be determined by titration and may also be given in the unit mg of KOH/g.

Hair treatment agents preferred as contemplated herein are characterized in that, based on their weight, they contain about 0.01 to about 20% by weight, preferably about 0.1 to about 10% by weight, more preferably about 0.5 to about 7.5% by weight, and in particular about 1 to about 5% by weight of amino-functional silicone(s).

The agents contemplated herein contain with particular preference amino-functional silicone(s) with terminal hydroxy group(s). Some special amino-functional silicone(s) with terminal hydroxy group(s) have emerged as being especially suitable in the agents contemplated herein. These are described hereafter.

Agents preferred as contemplated herein are characterized in that, based on their weight, they contain about 0.01 to about 20% by weight, preferably about 0.1 to about 10% by weight, particularly preferably about 0.5 to about 7.5% by weight, and in particular about 1 to about 5% by weight of at least one silicone of the following formula (Si-III)

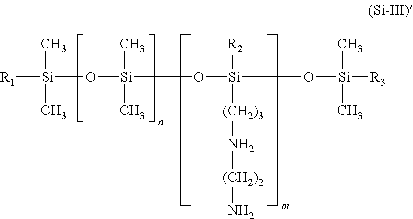

in which
m and n denote numbers that are selected such that the sum (n+m) is in the range of 1 to 1000,
n is a number in the range of 0 to 999 and m is a number in the range of 1 to 1000,
R1, R2, and R3, which are identical or different, denote a hydroxy group or a C1-4 alkoxy group,
at least one of the R1 to R3 groups denoting a hydroxy group.

Further agents preferred as contemplated herein are characterized in that, based on their weight, they contain about 0.01 to about 20% by weight, preferably about 0.1 to about 10% by weight, particularly preferably about 0.5 to about 7.5% by weight, and in particular about 1 to about 5% by weight of at least one silicone of the following formula (Si-IV)

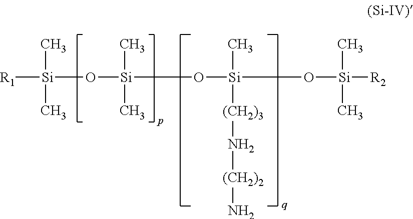

in which
p and q denote numbers that are selected such that the sum (p+q) is in the range of 1 to 1000,
p is a number in the range of 0 to 999 and q is a number in the range of 1 to 1000,
R1 and R2, which are different, denote a hydroxy group or a C1-4 alkoxy group, at least one of the R1 to R2 groups denoting a hydroxy group.

The silicones of the formulas (Si-III) and (Si-IV) differ by the grouping on the Si atom that carries the nitrogen-containing group: In formula (Si-III), R2 denotes a hydroxy group or a C1-4 alkoxy group, whereas the group in formula (Si-IV) is a methyl group. The individual Si groupings, which are labeled with the subscripts m and n or p and q, need not be present as blocks, but rather the individual units can also be distributed randomly; i.e., in the formulas (Si-III) and (Si-IV) each R1-Si(CH₃)₂ group is not necessarily bound to a —[O—Si(CH₃)₂] grouping.

Pretreatment agents that contain at least one silicone of the formula (Si-V) have emerged as especially effective in the method contemplated herein in regard to the desired effects:

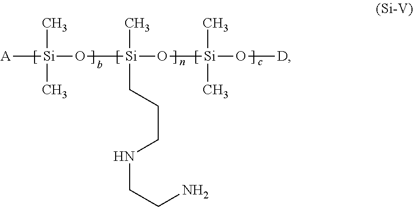

in which

A stands for a group —OH, —O—Si(CH₃)₃, —O—Si(CH₃)₂OH, —O—Si(CH₃)₂OCH₃,

D stands for a group —H, —Si(CH₃)₃, —Si(CH₃)₂OH, —Si(CH₃)₂OCH₃,

b, n and c stand for integers between 0 and 1000,
provided that
   n>0 and b+c>0
   at least one of the conditions A=—OH or D=—H is met.

Hair treatment agents contemplated herein that contain, based on their weight, about 0.01 to about 20% by weight, preferably about 0.1 to about 10% by weight, particularly preferably about 0.5 to about 7.5% by weight, and in particular about 1 to about 5% by weight of at least one silicone of the formula (Si-V):

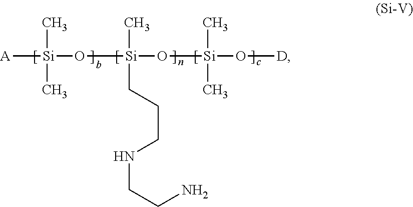

in which

A stands for a group —OH, —O—Si(CH₃)₃, —O—Si(CH₃)₂OH, —O—Si(CH₃)₂OCH₃,

D stands for a group —H, —Si(CH₃)₃, —Si(CH₃)₂OH, —Si(CH₃)₂OCH₃,

b, n and c stand for integers between 0 and 1000,
provided that
   n>0 and b+c>0
   at least one of the conditions A=—OH or D=—H is met,

are accordingly preferred as contemplated herein.

In the aforementioned formula (Si-V), the individual siloxane units with the subscripts b, c, and n are randomly distributed; i.e., they need not necessarily be block copolymers.

Further particularly suitable silicones are 4-morpholinomethyl-substituted. Hair treatment agents contemplated herein are particularly preferred that contain, based on their weight, about 0.01 to about 20% by weight, preferably about 0.1 to about 10% by weight, particularly preferably about 0.5 to about 7.5% by weight, and in particular about 1 to about 5% by weight of at least one 4-morpholinomethyl-substituted silicone of the formula (Si-VI),

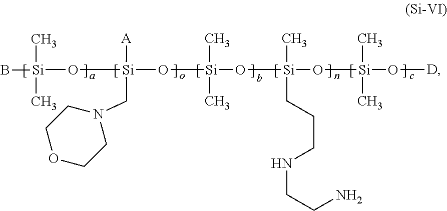

in which

A stands for structural unit (i) bound via an —O—

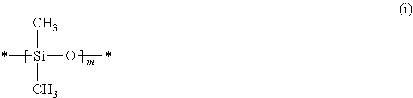

or for an oligomeric or polymeric group bound via an —O— and containing structural units of the formula (i) or for —OH,

* stands for a bond to the structural unit (i) or for an end group B (Si-bound) or D (O-bound),

B stands for a group —OH, —O—Si(CH₃)₃, —O—Si(CH₃)₂OH, —O—Si(CH₃)₂OCH₃,

D stands for a group —H, —Si(CH₃)₃, —Si(CH₃)₂OH, —Si(CH₃)₂OCH₃,

a, b and c stand for integers between 0 and 1000, with the proviso that a+b+c>0

m, n, and o stand for integers between 1 and 1000,

with the proviso that at least one of the conditions B=—OH or D=—H is met.

Structural formula (Si-VI) is intended to clarify that the siloxane groups n and o need not necessarily be bound directly to an end grouping B or D. Instead, in preferred formulas (Si-VI) a>0 or b>0 and in particularly preferred formulas (Si-VI) a>0 and b>0; i.e., the terminal grouping B or D is preferably bound to a dimethylsiloxy grouping. In formula (Si-VI) as well, the siloxane units a, b, c, n, and o are preferably distributed randomly.

The silicones represented by formula (Si-VI) and used as contemplated herein can be trimethylsilyl-terminated (D or B=—Si(CH₃)₃), but they may also be dimethylsilylhydroxy-terminated at both ends or dimethylsilylhydroxy- and dimethylsilylmethoxy-terminated at one end. In the context as contemplated herein, silicones used with particular preference are selected from silicones in which

B=—O—Si(CH₃)₂OH and D=—Si(CH₃)₃

B=—O—Si(CH₃)₂OH and D=—Si(CH₃)₂OH

B=—O—Si(CH₃)₂OH and D=—Si(CH₃)₂OCH₃

B=—O—Si(CH₃)₃ and D=—Si(CH₃)₂OH

B=—O—Si(CH₃)₂OCH₃ and D=—Si(CH₃)₂OH.

These silicones lead to enormous improvements in the hair properties of hair treated with the agents contemplated herein, in particular to greatly improved protection during oxidative treatment.

In formula (Si-VI) as well, the A group can stand
for a structural unit (i) bound via an —O— or
an oligomeric or polymeric group bound via an —O— and containing structural units of the formula (i)
or for —OH.

Thus, formula (Si-VI) is more precisely one of the formulas (Si-VIa), (Si-VIb), or (Si-VIc):

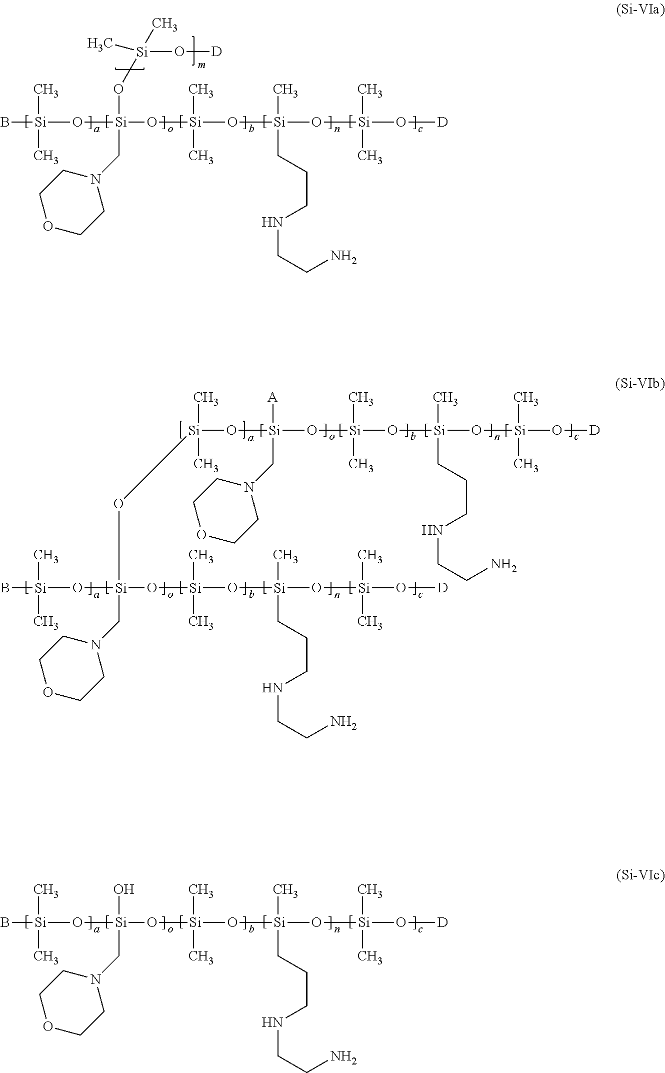

Irrespective of the type of employed amino-functional silicone(s) with terminal hydroxy group(s), the agents contemplated herein contain the silicone(s) preferably in the form of an emulsion, particularly preferably in the form of a microemulsion.

It has been found that the action of the silicones used in the agents contemplated herein can be increased still further if specific nonionic components are also used in the agents. In addition, these nonionic components have positive effects on the storage stability of the agents. Nonionic components that are particularly suitable here are ethoxylates of decanol, undecanol, dodecanol, tridecanol, etc. Ethoxylated tridecanols which are incorporated more preferentially into the agents contemplated herein have proven particularly suitable. Particularly preferred agents as contemplated herein are characterized in that, based on their weight, they contain about 0.00001 to about 5% by weight, preferably about 0.0001 to about 3.5% by weight, particularly preferably about 0.001 to about 2% by weight, more preferably about 0.01 to about 1% by weight, and in particular about 0.1 to about 0.5% by weight of branched, ethoxylated tridecanol (INCI name: Trideceth-5) or α-isotridecyl-ω-hydroxypolyglycolether (INCI name: Trideceth-10), or mixtures thereof.

The hair treatment agents contain at least one organic acid.

Typical representatives of organic acids are aliphatic mono- and dicarboxylic acids such as, for example, acetic acid, propionic acid, oxalic acid, and 1,3-propanedioic acid and aromatic carboxylic acids such as, for example, benzoic acid. Other organic acids are, for example, hydroxycarboxylic acids such as glycolic acid, citric acid, tartaric acid, malic acid, and lactic acid. Unsaturated mono- or dicarboxylic acids such as, for example, fumaric acid, or α-ketocarboxylic acids such as, for example, pyruvic acid (2-oxopropionic acid) are included as contemplated herein.

Hair treatment agents preferred as contemplated herein contain, based on their weight, about 0.001 to about 10% by weight, preferably about 0.01 to about 5% by weight, more preferably about 0.05 to about 3% by weight, and in particular about 0.1 to about 2% by weight of organic acid(s).

Particularly preferred hair treatment agents contain, based on their weight, about 0.001 to about 10% by weight, preferably about 0.01 to about 5% by weight, more preferably about 0.05 to about 3% by weight, and in particular about 0.1 to about 2% by weight of organic acid(s) from the group comprising formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, dicarbamic acid, 4,4′-dicyano-6,6′-dinicotinic acid, 8-carbamoyloctanoic acid, 1,2,4-pentanetricarboxylic acid, 2-pyrrolecarboxylic acid, 1,2,4,6,7-naphthalenepentaacetic acid, malonaldehydic acid, 4-hydroxyphthalamidic acid, 1-pyrazolecarboxylic acid, gallic acid, or propanetricarboxylic acid glycolic acid, lactic acid, malic acid, tartaric acid, or citric acid.

Some representatives are particularly preferred from among the aforementioned acids, because they impart in addition a pleasant shine to hair treated with agents contemplated herein. Most preferred hair treatment agents contain, based on their weight, about 0.001 to about 10% by weight, preferably about 0.01 to about 5% by weight, more preferably about 0.05 to about 3% by weight, and in particular about 0.1 to about 2% by weight of organic acid(s) from the group comprising formic acid, oxalic acid, maleic acid, lactic acid, tartaric acid, or citric acid.

The hair treatment agents contain phenoxyisopropanol (1-phenoxypropan-2-ol). Phenoxyisopropanol is preferably used within defined narrow amount ranges. Here hair treatment agents contemplated herein are preferred that contain, based on their weight, about 0.01 to about 3% by weight, preferably about 0.025 to about 2.5% by weight, more preferably about 0.05 to about 2% by weight, and in particular about 0.1 to about 1% by weight of phenoxyisopropanol.

The hair treatment agents contain the above-described active substances preferably in a cosmetic acceptable carrier. In the context contemplated herein, this is understood to be preferably an aqueous or aqueous-alcoholic carrier.

The cosmetic carrier contains preferably at least about 50% by weight, more preferably at least about 60% by weight, particularly preferably at least about 70% by weight, and especially preferably at least about 75% by weight of water.

Furthermore, the cosmetic carrier can contain about 0.01 to about 40% by weight, preferably about 0.05 to about 30% by weight, and in particular about 0.1 to about 20% by weight of at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, phenoxyethanol, or mixtures of said alcohols.

The water-soluble alcohols are particularly preferred. Ethanol, 1,2-propylene glycol, glycerol, benzyl alcohol, and mixtures of said alcohols are particularly preferred.

It is of advantage for a very good (scalp) skin compatibility of the hair treatment agents contemplated herein if these have a slightly acidic pH.

It has been found that the agents contemplated herein have an especially good skin compatibility and mildness in a pH range of 4.2 to 5.8.

In a first preferred embodiment, the hair treatment agents contemplated herein therefore preferably have a pH in the range of 4.2 to 5.8, more preferably from 4.25 to 5.6, particularly preferably from 4.3 to 5.5, exceedingly preferably from 4.35 to 5.4, and especially preferably from 4.4 to 5.3.

The hair treatment agents contemplated herein may contain vegetable oils, vegetable butters, and/or vegetable waxes. These vegetable oil components give the hair an improved combability and stylability and increase the hair shine.

Suitable vegetable oil components include natural (vegetable) oils and/or butter, which typically contain triglycerides and mixtures of triglycerides.

Preferred natural oils are coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Carnauba wax, beeswax, and/or candelilla wax can be used with preference as suitable natural or vegetable waxes.

Particularly preferred vegetable oil components are (sweet) almond oil, peach kernel oil, apricot kernel oil, amaranth seed oil, argan oil, olive oil, jojoba oil, cocoa butter, and/or shea butter.

Apricot kernel oil, argan oil, olive oil, and/or jojoba oil are particularly preferred.

In a preferred embodiment, the hair treatment agents contemplated herein preferably contain coconut oil, (sweet) almond oil, walnut oil, peach kernel oil, apricot kernel oil, argan oil, avocado oil, tea tree oil, soybean oil, sesame oil, sunflower oil, tsubaki oil, evening primrose oil, rice bran oil, palm kernel oil, mango kernel oil, marula oil, cuckoo flower oil, thistle oil, macadamia nut oil, grape seed oil, amaranth seed oil, bamboo oil, olive oil, wheat germ oil, pumpkin seed oil, mallow oil, hazelnut oil, safflower oil, canola oil, sasanqua oil, jojoba oil, rambutan oil, cocoa butter, and/or shea butter.

Within this embodiment, it is particularly preferable if the hair treatment agents contemplated herein contain (sweet) almond oil, peach kernel oil, apricot kernel oil, amaranth seed oil, olive oil, jojoba oil, cocoa butter, and/or shea butter.

The weight proportion of the at least one vegetable oil, vegetable butter, and/or vegetable wax in terms of the total weight of the hair treatment agents contemplated herein is preferably about 0.02 to about 2.50% by weight, more preferably of about 0.03 to about 2.00% by weight, particularly preferably about 0.04 to about 1.50% by weight, and in particular about 0.05 to about 1.00% by weight.

Apart from the aforementioned essential and facultative components, the hair treatment agents contemplated herein in a further preferred embodiment for a further increase in the care properties of the agents can contain at least one further active hair-conditioning substance, which can be selected from the group of
protein hydrolysates,
vitamins,
plant extracts, and/or
glycerol.

Suitable protein hydrolysates are understood to be product mixtures that can be obtained by acid-, base-, or enzyme-catalyzed degradation of proteins.

Protein hydrolysates of plant, animal, and/or marine origin can be used.

Animal protein hydrolysates are, for example, elastin, collagen, keratin, silk, and milk protein hydrolysates, which can also be present in the form of salts. Such products are marketed, for example, under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex), and Kerasol® (Croda).

Preferred are protein hydrolysates of plant origin, e.g., soybean, almond, rice, pea, potato, and wheat protein hydrolysates. Such products are available, for example, under the tradenames Gluadin® (Cognis), DiaMin® (Diamalt), Lexein® (Inolex), and Crotein® (Croda). Cationized protein hydrolysates can also be used, wherein the underlying protein hydrolysate may originate from animals, for example, from collagen, milk, or keratin, from plants, for example, from wheat, corn, rice, potatoes, soybeans, or almonds, from marine life forms, for example, from fish collagen or algae, or from biotechnologically obtained protein hydrolysates. The protein hydrolysates underlying the cationic derivatives may be obtained from the corresponding proteins by chemical, in particular, alkaline or acidic hydrolysis, by enzymatic hydrolysis, and/or by a combination of both types of hydrolysis. The hydrolysis of proteins usually gives rise to a protein hydrolysate with a molecular weight distribution of about 100 Da up to several thousand daltons. Preferred cationic protein hydrolysates are those whose underlying protein portion has a molecular weight of about 100 up to about 25,000 Da, preferably about 250 to about 5000 Da. Furthermore, cationic protein hydrolysates are understood to be quaternized amino acids and mixtures thereof. Quaternization of the protein hydrolysates or of the amino acids is often performed by means of quaternary ammonium salts such as, for example, N,N-dimethyl-N-(n-alkyl)-N-(2-hydroxy-3-chloro-n-propyl)ammonium halides. Furthermore, the cationic protein hydrolysates can also be derivatized still further. Mentioned as typical examples of the cationic protein hydrolysates and derivatives are the products that are commercially available and are known under the INCI names: Cocodimonium Hydroxypropyl Hydrolyzed Collagen, Cocodimopnium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, and Quaternium-79 Hydrolyzed Wheat Protein.

The weight proportion of the protein hydrolysate(s) in terms of the total weight of the hair treatment agents is preferably about 0.01 to about 5% by weight, more preferably about 0.025 to about 3% by weight, and in particular about 0.05 to about 2% by weight.

Irrespective of the source (plant, animal, marine, etc.), protein hydrolysates depending on the degree of hydrolysis contain individual amino acids, oligopeptides, and optionally polypeptides.

The hair treatment agents contemplated herein contain with particular preference at least one oligopeptide, which has at least one amino acid sequence Glu-Glu-Glu

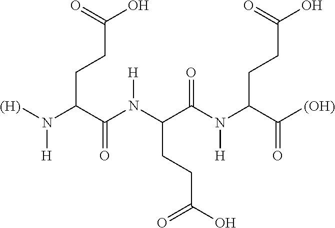

wherein the amino group may be present in a free or protonated form and the carboxy groups in a free or deprotonated form.

Preferred hair treatment agents contemplated herein are characterized in that, based on their weight, they contain about 0.0001 to about 10% by weight of at least one oligopeptide, which has at least one amino acid sequence Glu-Glu-Glu

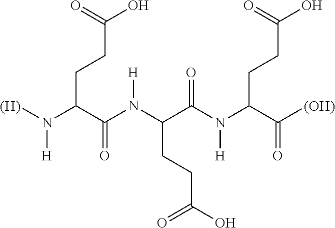

wherein the amino group may be present in a free or protonated form and the carboxy groups in a free or deprotonated form.

In this formula, as in all formulas below, the amino group hydrogen atom in parentheses like the acid function hydroxy group in parentheses means that the groups in question may be present as such (in which case it is an oligopeptide with the particular number of amino acids as shown (in the present formula 3)) or, however, that the amino acid sequence is present in an oligopeptide that comprises still further amino acids (depending on where the further amino acid(s) is/are bound, the components in parentheses in the above formula are replaced by the further amino acid group(s)).

These preferred hair treatment agents contemplated herein contain, based on their weight, about 0.0001 to about 10% by weight of at least one oligopeptide that has at least one amino acid sequence Glu-Glu-Glu, i.e., at least three consecutive glutamic acids.

Oligopeptides in the context of the present application are amino acid condensation products which are linked acid amide-like by peptide bonds and comprise at least 3 and a maximum of 25 amino acids.

In preferred hair treatment agents contemplated herein, the oligopeptide comprises 5 to 15 amino acids, preferably 6 to 13 amino acids, particularly preferably 7 to 12 amino acids, and in particular 8, 9 or 10 amino acids.

Depending on whether additional amino acids are bound to the Glu-Glu-Glu sequence and depending on the type of said amino acids, the molar mass of the oligopeptide present in the agents contemplated herein may vary. Preferred hair treatment agents used as contemplated herein are characterized in that the oligopeptide has a molar mass of about 650 to about 3000 Da, preferably of about 750 to about 2500 Da, particularly preferably of about 850 to about 2000 Da, and in particular of about 1000 to about 1600 Da.

In summary, preferred hair treatment agents are characterized in that the oligopeptide comprises 5 to 15 amino acids, preferably 6 to 13 amino acids, particularly preferably 7 to 12 amino acids, and in particular 8, 9, or 10 amino acids and has a molar mass of about 650 to about 3000 Da, preferably of about 750 to about 2500 Da, particularly preferably of about 850 to about 2000 Da, and in particular of about 1000 to about 1600 Da.

As can be seen from the preferred number of amino acids in the oligopeptides and the preferred molar mass range, oligopeptides consisting not only of the three glutamic acids but also having additional amino acids bound to this sequence are used with preference. These additional amino acids are preferably selected from specific amino acids, whereas specific other representatives are less preferred as contemplated herein.

It is thus preferred, if the oligopeptides used in the agents contemplated herein do not contain any methionine.

It is preferred further, if the oligopeptides used in the agents contemplated herein do not contain any cysteine and/or cystine.

It is preferred further, if the oligopeptides used in the agents contemplated herein do not contain any aspartic acid and/or asparagine.

It is preferred further, if the oligopeptides used in the agents contemplated herein do not contain any serine and/or threonine.

In contrast, it is preferred, if the oligopeptides used in the agents contemplated herein contain tyrosine.

It is preferred further, if the oligopeptides used in the agents contemplated herein contain leucine.

It is preferred further, if the oligopeptides used in the agents contemplated herein contain isoleucine.

It is preferred further, if the oligopeptides used in the agents contemplated herein contain arginine.

It is preferred further, if the oligopeptides used in the agents contemplated herein contain valine.

Particularly preferred oligopeptides or amino acid sequences contained in the preferred oligopeptides are described hereafter:

A particularly preferred oligopeptide additionally contains tyrosine, which is bound preferably via its acid function to the Glu-Glu-Glu sequence. Hair treatment agents preferred as contemplated herein are therefore characterized in that the oligopeptide contained therein has at least one amino acid sequence Tyr-Glu-Glu-Glu

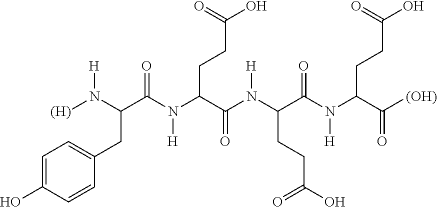

wherein the amino group may be present in a free or protonated form and the carboxy groups in a free or deprotonated form.

A further particularly preferred oligopeptide additionally contains isoleucine, which is bound preferably via its amino function to the Glu-Glu-Glu sequence. Hair treatment agents preferred as contemplated herein are therefore characterized in that the oligopeptide contained therein has at least one amino acid sequence Glu-Glu-Glu-Ile

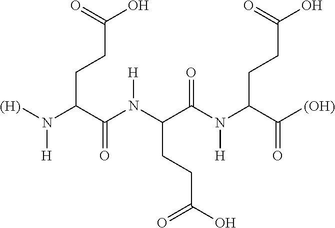

wherein the amino group may be present in a free or protonated form and the carboxy groups in a free or deprotonated form.

Oligopeptides that have both of the aforesaid amino acids (tyrosine and isoleucine) are preferred as contemplated herein. Particularly preferred in this case are hair treatment agents contemplated herein in which the oligopeptide contained in the hair treatment agent has at least one amino acid sequence Tyr-Glu-Glu-Glu-Ile

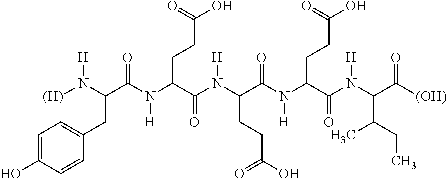

wherein the amino group may be present in a free or protonated form and the carboxy groups in a free or deprotonated form.

Further preferred oligopeptides additionally contain arginine, which is preferably present bound to isoleucine.

Particularly preferred in this case are hair treatment agents contemplated herein in which the oligopeptide contained in the hair treatment agent has at least one amino acid sequence Tyr-Glu-Glu-Glu-Ile-Arg

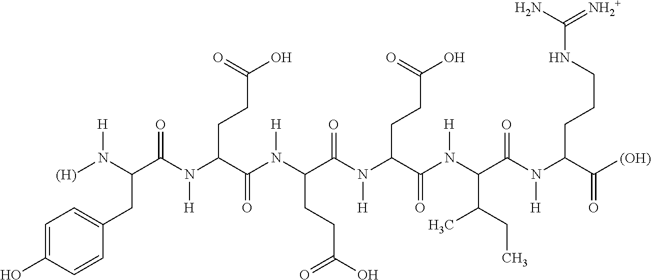

wherein the amino groups may be present in a free or protonated form and the carboxy groups in a free or deprotonated form.

Still further preferred oligopeptides additionally contain valine, which is preferably present bound to arginine. Hair treatment agents preferred further as contemplated herein are therefore characterized in that the oligopeptide contained in the hair treatment agent has at least one amino acid sequence Tyr-Glu-Glu-Glu-Ile-Arg-Val

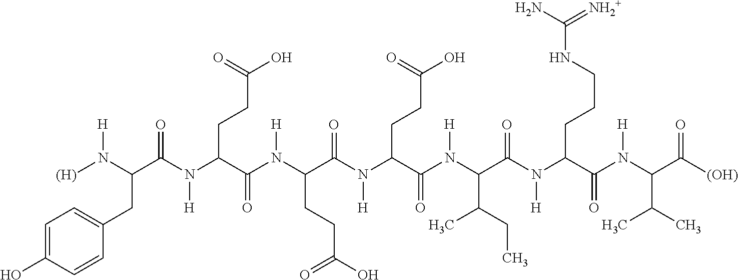

wherein the amino groups may be present in a free or protonated form and the carboxy groups in a free or deprotonated form.

Still further preferred oligopeptides additionally contain leucine, which is preferably present bound to valine. Hair treatment agents preferred further as contemplated herein are therefore characterized in that the oligopeptide contained in the hair treatment agent has at least one amino acid sequence Tyr-Glu-Glu-Glu-Ile-Arg-Val-Leu

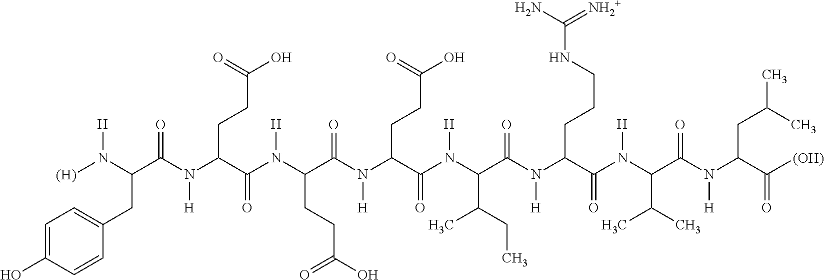

wherein the amino groups may be present in a free or protonated form and the carboxy groups in a free or deprotonated form.

Particularly preferred oligopeptides additionally contain leucine, which is preferably present bound to tyrosine. Hair treatment agents preferred further as contemplated herein are therefore characterized in that the oligopeptide contained in the hair treatment agent has at least one amino acid sequence Leu-Tyr-Glu-Glu-Glu-Ile-Arg-Val-Leu

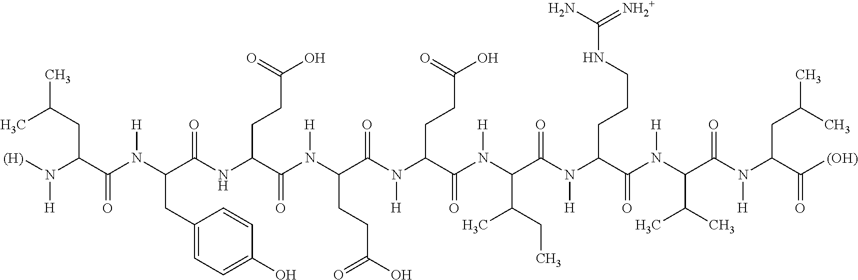

wherein the amino groups may be present in a free or protonated form and the carboxy groups in a free or deprotonated form.

Very particularly preferably, the agents contemplated herein contain at least two oligopeptides that meet the aforesaid criteria, but differ from one another. Thus, for example, the use of hair treatment agents is preferred that contain at least two different oligopeptides A and B, both of which contain the amino acid sequence Glu-Glu-Glu.

Such different oligopeptides A and B correspond to one another in that they have three consecutive Glu amino acids in their amino acid sequence, but have differences in the amino acids bound before or after these. Different peptides with a partial agreement, which may be much greater than in the aforementioned three amino acids, are preferred.

Thus, further preferred hair treatment agents are characterized in that the hair treatment agent contain at least two different oligopeptides A and B, both of which contain the amino acid sequence Glu-Glu-Glu-Ile.

Also preferred are hair treatment agents that contain at least two different oligopeptides A and B, both of which contain the amino acid sequence Tyr-Glu-Glu-Glu.

Even further preferred hair treatment agents are characterized in that they contain at least two different oligopeptides A and B, both of which contain the amino acid sequence Glu-Glu-Glu-Ile-Arg.

Likewise, even further preferred hair treatment agents are characterized in that the hair treatment agents contain at least two different oligopeptides A and B, both of which contain the amino acid sequence Tyr-Glu-Glu-Glu-Ile.

Preferred hair treatment agents contemplated herein are therefore characterized in that the oligopeptide has at least one amino acid sequence Tyr-Glu-Glu-Glu-Ile

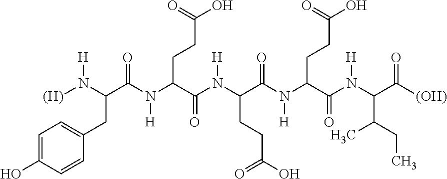

wherein the amino group may be present in a free or protonated form and the carboxy groups in a free or deprotonated form.

Very particularly preferred hair treatment agents are characterized in that the hair treatment agent contains at least two different oligopeptides A and B, both of which contain the amino acid sequence Glu-Glu-Glu-Ile-Arg.

Likewise, very particularly preferred hair treatment agents are characterized in that the hair treatment agent contains at least two different oligopeptides A and B, both of which contain the amino acid sequence Tyr-Glu-Glu-Glu-Ile-Arg.

Preferably, there is an even greater structural agreement in the oligopeptides. Thus, hair treatment agents that contain at least two different oligopeptides A and B, both of which contain the amino acid sequence Glu-Glu-Glu-Ile-Arg-Val, are further preferred embodiments as contemplated herein.

Likewise, preferred embodiments are hair treatment agents that contain at least two different oligopeptides A and B, both of which contain the amino acid sequence Tyr-Glu-Glu-Glu-Ile-Arg-Val.

Even further preferred hair treatment agents contemplated herein are characterized in that they contain at least two different oligopeptides A and B, both of which contain the amino acid sequence Glu-Glu-Glu-Ile-Arg-Val-Leu.

Likewise, still further preferred hair treatment agents contemplated herein are characterized in that they contain at least two different oligopeptides A and B, both of which contain the amino acid sequence Tyr-Glu-Glu-Glu-Ile-Arg-Val-Leu.

Preferred hair treatment agents contemplated herein are therefore characterized in that the oligopeptide has at least one amino acid sequence Tyr-Glu-Glu-Glu-Ile-Arg-Val-Leu

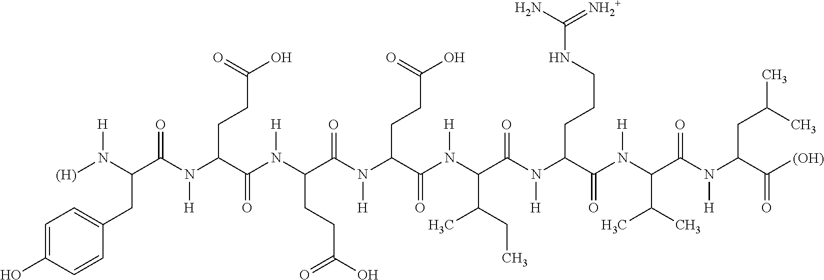

wherein the amino groups may be present in a free or protonated form and the carboxy groups in a free or deprotonated form,

Particularly preferred hair treatment agents contemplated herein are characterized in that they contain at least two different oligopeptides A and B, wherein the oligopeptide A has the amino acid sequence Leu-Tyr-Glu-Glu-Glu-Ile-Arg-Val-Leu

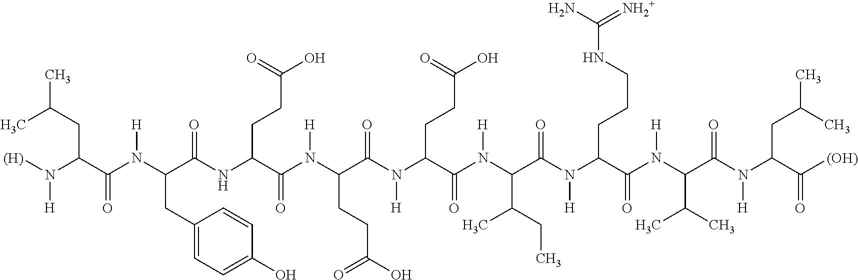

wherein the amino groups may be present in a free or protonated form and the carboxy groups in a free or deprotonated form,
and the oligopeptide B has the amino acid sequence Tyr-Glu-Glu-Glu-Ile-Arg-Val-Leu

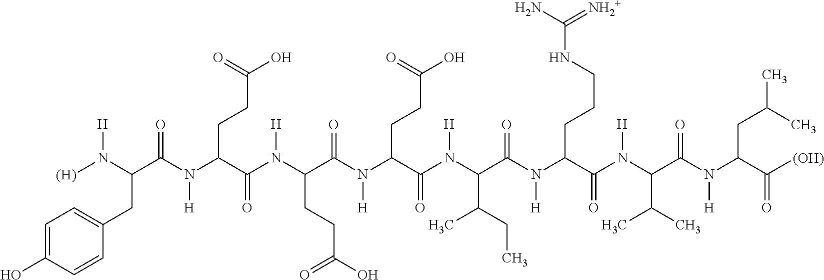

wherein the amino groups may be present in a free or protonated form and the carboxy groups in a free or deprotonated form.

The very particularly preferred hair treatment agents of this latter embodiment contain, based on the weight of the agent, about 0.00001 to about 1% by weight of oligopeptide A and about 0.00001 to about 1% by weight of oligopeptide B.

The further preferred hair treatment agents of this latter embodiment contain, based on the weight of the agent, about 0.00005 to about 0.1% by weight of oligopeptide A and about 0.00005 to about 0.1% by weight of oligopeptide B.

The still further preferred hair treatment agents of this latter embodiment contain, based on the weight of the agent, about 0.0001 to about 0.01% by weight of oligopeptide A and about 0.0001 to about 0.001% by weight of oligopeptide B.

The oligopeptides used in the context as contemplated herein, which meet the aforesaid conditions, can be obtained advantageously from keratinic materials. It is preferred as contemplated herein that these oligopeptides are used in large amounts, based on the total peptide content of the agents.

It is very particularly preferred that the greatest possible amount of all keratinic peptides contained in the agent contemplated herein meets the aforesaid conditions.

Preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Glu-Glu-Glu.

Additionally, preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Glu-Glu-Glu-Ile.

Even more preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Tyr-Glu-Glu-Glu.

Particularly preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Tyr-Glu-Glu-Glu-Ile.

Very particularly preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Tyr-Glu-Glu-Glu-Ile-Arg.

Even more particularly preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Tyr-Glu-Glu-Glu-Ile-Arg-Val.

Particularly preferred hair treatment agents contemplated herein are characterized in that at least about 0.1% by weight, preferably at least about 0.5% by weight, particularly preferably at least about 1% by weight, more preferably at least about 2.5% by weight, even more preferably at least about 5% by weight, and in particular at least about 10% by weight of all keratinic peptides, contained in the agent, have the amino acid sequence Tyr-Glu-Glu-Glu-Ile-Arg-Val-Leu.

The aforesaid conditions relate to the total content of peptides, originating from keratinic materials, in the agents contemplated herein. In addition to the oligopeptides of keratinic origin, naturally further peptides and/or protein hydrolysates can be used, for example, from other native sources. For example, the additional use of wheat protein hydrolysates is preferred.

Suitable vitamins are understood preferably as the following vitamins, provitamins, and vitamin precursors, and derivatives thereof:
Vitamin A: the group of substances designated as vitamin A include retinol (Vitamin A₁) and 3,4-didehydroretinol (vitamin A₂). β-Carotene is the retinol provitamin. Suitable vitamin A components are, for example, vitamin A acid and esters thereof, vitamin A aldehyde, and vitamin A alcohol and esters thereof such as the palmitate and the acetate.
Vitamin B: The vitamin B group or the vitamin B complex includes, inter alia,
   vitamin B₁ (thiamine)
   vitamin B₂ (riboflavin)
   vitamin B₃. The compounds nicotinic acid and nicotinamide (niacinamide) are often included under this term.
   vitamin B₅ (pantothenic acid and panthenol). Panthenol is preferably used within the scope of this group. Usable panthenol derivatives are particularly the esters and ethers of panthenol, pantolactone, and cationically derivatized panthenols. Individual representatives are, for example, panthenol triacetate, panthenol monoethyl ether, and the monoacetate thereof, as well as cationic panthenol derivatives.
   vitamin B₆ (pyridoxine as well as pyridoxamine and pyridoxal).
Vitamin C (ascorbic acid): The use in the form of the palmitic acid ester, glucosides, or phosphates can be preferred. The use in combination with tocopherols can likewise be preferred.
Vitamin E (tocopherols, especially α-tocopherol).
Vitamin F: The term “vitamin F” is conventionally understood to mean essential fatty acids, in particular linoleic acid, linolenic acid, and arachidonic acid.
Vitamin H: Vitamin H is the name for the compound (3aS,4S,6aR)-2-oxohexahydrothienol[3,4-d]-imidazole-4-valeric acid, although the trivial name biotin has become accepted in the meantime.

Vitamins, provitamins, and vitamin precursors from the groups A, B, E, and H are particularly preferred. Preferred in particular are nicotinamide, biotin, pantolactone, and/or panthenol.

The weight proportion of the vitamin(s), vitamin derivative(s), and/or the vitamin precursor(s) in terms of the total weight of the hair treatment agents is preferably about 0.001 to about 2% by weight, particularly preferably about 0.005 to about 1% by weight, and in particular about 0.01 to about 0.5% by weight.

Extracts that can be prepared from all parts of a plant are to be understood as suitable plant extracts. These extracts are typically produced by extraction of the entire plant. It can also be preferred in individual cases, however, to produce the extracts solely from the flowers and/or leaves of the plant. Suitable above all are the extracts from green tea, oak bark, stinging nettle, witch hazel, hops, chamomile, burdock, horsetail, whitethorn, lime blossom, lychee, almond, aloe vera, spruce needles, horse chestnut, sandalwood, juniper, coconut, mango, apricot, lemon, wheat, kiwi, melon, orange, grapefruit, sage, rosemary, birch, mallow, cuckoo flower, wild thyme, yarrow, thyme, melissa, restharrow, coltsfoot, marshmallow, ginseng, ginger root, Echinacea purpurea, Olea europaea, Boerhavia diffusa roots, Foeniculum vulgaris, and Apium graveolens.

The extracts from green tea, stinging nettle, witch hazel, chamomile, aloe vera, ginseng, Echinacea purpurea, Olea europea, and/or Boerhavia diffusa roots are particularly preferred for use in the compositions contemplated herein.

Water, alcohols, and mixtures thereof can be used as extracting agents to produce the cited plant extracts. Of the alcohols, low alcohols such as ethanol and isopropanol, but in particular polyhydric alcohols such as ethylene glycol and propylene glycol, are preferred, both as the sole extracting agent and in a mixture with water. Plant extracts based on water/propylene glycol in the ratio of 1:10 to 10:1 have proven to be particularly suitable.

The plant extracts can be used both in pure and diluted form. If they are used in diluted form, they conventionally contain about 2 to about 80% by weight of active substance and as the solvent, the extracting agent or mixture of extracting agents used to obtain them.

The plant extracts can be used in the hair treatment agents contemplated herein (based on the total weight of the agents) preferably in an amount of about 0.01 to about 10% by weight, more preferably of about 0.05 to about 7.5% by weight, and in particular of about 0.1 to about 5% by weight.

Glycerol can be added to the hair cleaning and care agents separately in an amount of up to about 10% by weight (based on the total weight of the agent). However, it can also be a component of the aforesaid aqueous-alcoholic carrier.

It was found that the hair treatment agents contemplated herein are also suitable for use as an anti-dandruff formulation.

The total weight of anti-dandruff agents in terms of the total weight of the hair treatment agents can preferably be about 0.01 to about 10% by weight, more preferably about 0.025 to about 7.5% by weight, particularly preferably about 0.05 to about 5% by weight, and in particular about 0.075 to about 3% by weight.

Suitable anti-dandruff active agents can be selected from piroctone olamine, climbazole, zinc pyrithione, ketoconazole, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts, and/or arnica extracts.

Climbazole, zinc pyrithione, and piroctone olamine are preferred.

Other active substances, auxiliary substances, and additives that can be contained in the hair treatment agents contemplated herein are, for example:
humectants,
perfume,
UV filters,
thickeners, such as gelatin or plant gum, for example, agar-agar, guar gum, alginates, xanthan gum, gum arabic, karaya gum, locust bean gum, flaxseed gums, dextrans, cellulose derivatives, e.g., methyl cellulose, hydroxyalkyl cellulose, and carboxymethyl cellulose, starch fractions and derivatives such as amylose, amylopectin, and dextrins, clays and phyllosilicates such as, e.g., bentonite, or fully synthetic hydrocolloids such as, e.g., polyvinyl alcohol, Ca, Mg, or Zn soaps,
structurants such as maleic acid and lactic acid,
dimethyl isosorbide,
cyclodextrins,
fiber-structure-improving active substances, particularly mono-, di-, and oligosaccharides such as, for example, glucose, galactose, fructose, fruit sugar, and lactose,
dyes for coloring the agent,
active substances such as bisabolol and/or allantoin,
complexing agents such as EDTA, NTA, β-alaninediacetic acid, and phosphonic acids,
ceramides. Ceramides are understood to be N-acylsphingosine (fatty acid amides of sphingosine) or synthetic analogs of such lipids (so-called pseudo-ceramides),
propellants such as propane-butane mixtures, N₂O, dimethyl ether, CO₂, and air,
antioxidants,
additional viscosity regulators such as salts (NaCl).

The agents contemplated herein are preferably so-called rinse-off products, i.e., are rinsed out of the hair again after a specific contact time. This contact time is preferably less than an hour; i.e., the user preferably does not leave the products in the hair until the next hair washing.

A further subject as contemplated herein is therefore a method for hair treatment in which an agent contemplated herein is applied to dry or damp hair, is left there for a time period of about 30 to about 300 seconds, and is rinsed out thereafter.

The agents contemplated herein lead to a considerably increased stability of artificial colors to the washing out of the color. Thus, chemically dyed hair can be washed much more often with the agents contemplated herein, without resulting in the unwanted bleeding or fading of the color.

A further subject as contemplated herein, therefore, is the use of the agents contemplated herein for reducing the washing out of color from chemically dyed hair.

The statements made about the agents contemplated herein apply mutatis mutandis in regard to preferred embodiments of the method contemplated herein and the use contemplated herein.

### Examples

All quantities are given in % by weight

Hair shampoo Series 1

| | 1 | 2 | 3 | 4 | 5 | 6 |
|---|---|---|---|---|---|---|
| Sodium Laureth Sulfate (AS) | 10.0 | 10.0 | 10.0 | 10.0 | 10.0 | 10.0 |
| Cocamidopropyl Betaine (AS) | 4.0 | 4.0 | 4.0 | 4.0 | 4.0 | 4.0 |
| Ammonium Lauryl Sulfate | 4.0 | 4.0 | 4.0 | 4.0 | 4.0 | 4.0 |
| Polyguaternium-10 | 0.3 | 0.3 | 0.3 | 0.3 | 0.3 | 0.3 |
| Cocamide MEA (AS) | 0.8 | 0.8 | 0.8 | 0.8 | 0.8 | 0.8 |
| PEG-7 Glyceryl Cocoate | 1.0 | 1.0 | 1.0 | 1.0 | 1.0 | 1.0 |
| Amodimethicone | 0.8 | 0.8 | 0.8 | 0.8 | 0.8 | 0.8 |
| Nicotinamide | 0.3 | 0.3 | 0.3 | 0.3 | 0.3 | 0.3 |
| Lactic acid | 0.2 | 0.2 | 0.2 | 0.2 | 0.2 | 0.2 |
| Panthenol | 0.5 | 0.5 | 0.5 | 0.5 | 0.5 | 0.5 |
| Laureth-2 | 1.2 | 1.2 | 1.2 | 1.2 | 1.2 | 1.2 |
| Sodium chloride | 1.3 | 1.3 | 1.3 | 1.3 | 1.3 | 1.3 |
| Phenoxyisopropanol | 0.5 | 0.5 | 0.5 | 0.5 | 0.5 | 0.5 |
| Ethyl Lauroyl Arginate | — | 0.4 | 0.3 | 0.2 | 0.1 | 0.05 |
| Glutaraldehyde | — | 0.05 | 0.06 | 0.07 | 0.08 | 0.1 |
| Dimethyloxazolidine | — | 0.05 | 0.06 | 0.07 | 0.08 | 0.1 |
| Phenoxyethanol | — | 0.05 | 0.05 | 0.05 | 0.05 | 0.05 |
| o-Phenylphenol | — | 1.0 | 0.5 | 0.25 | 0.2 | 0.1 |
| o-Cymen-5-ol | — | 0.1 | 0.05 | 0.1 | 0.05 | 0.1 |
| Hexetidine | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |

| | |
|---|---|
| Water | To 100 |

Hair shampoo Series 2

| | 1 | 2 | 3 | 4 | 5 | 6 |
|---|---|---|---|---|---|---|
| Sodium Laureth Sulfate | 7.0 | 7.0 | 7.0 | 7.0 | 7.0 | 7.0 |
| (AS) | | | | | | |
| Disodium | 4.0 | 4.0 | 4.0 | 4.0 | 4.0 | 4.0 |
| Cocoamphodiacetate (AS) | | | | | | |
| Coco Glucoside | 3.0 | 3.0 | 3.0 | 3.0 | 3.0 | 3.0 |
| Cocamide MEA (AS) | 0.3 | 0.3 | 0.3 | 0.3 | 0.3 | 0.3 |
| Polyquaternium-67 | 0.1 | 0.1 | 0.1 | 0.1 | 0.1 | 0.1 |
| Amodimethicone | 0.8 | 0.8 | 0.8 | 0.8 | 0.8 | 0.8 |
| Panthenol | 0.2 | 0.2 | 0.2 | 0.2 | 0.2 | 0.2 |
| Jojoba Seed Oil | 0.05 | 0.05 | 0.05 | 0.05 | 0.05 | 0.05 |
| Wheat protein hydrolysate | 0.3 | 0.3 | 0.3 | 0.3 | 0.3 | 0.3 |
| Citric acid | 0.4 | 0.4 | 0.4 | 0.4 | 0.4 | 0.4 |
| PEG-120 Methyl Glucose | 0.7 | 0.7 | 0.7 | 0.7 | 0.7 | 0.7 |
| Dioleate | | | | | | |
| Phenoxyisopropanol | 1.0 | 1.0 | 1.0 | 1.0 | 1.0 | 1.0 |
| Ethyl Lauroyl Arginate | — | 0.4 | 0.3 | 0.2 | 0.1 | 0.05 |
| Glutaraldehyde | — | 0.05 | 0.06 | 0.07 | 0.08 | 0.1 |
| Dimethyloxazolidine | — | 0.05 | 0.06 | 0.07 | 0.08 | 0.1 |
| Phenoxyethanol | — | 0.05 | 0.05 | 0.05 | 0.05 | 0.05 |
| Sodium-o-phenylphenolate | — | 1.0 | 0.5 | 0.25 | 0.2 | 0.1 |
| o-Cymen-5-ol | — | 0.1 | 0.05 | 0.1 | 0.05 | 0.1 |
| Hexetidine | — | 0.2 | 0.1 | 0.2 | 0.1 | 0.1 |
| Propionic acid | — | 0.05 | 0.1 | 0.25 | 0.05 | 0.05 |
| Undecylenic acid | — | 0.1 | 0.2 | 0.1 | 0.2 | 0.1 |
| Dye | 0.2 | 0.2 | 0.2 | 0.2 | 0.2 | 0.2 |
| Perfume | 0.1 | 0.1 | 0.1 | 0.1 | 0.1 | 0.1 |
| Water | To 100 | | | | | |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

1. A hair treatment agent, comprising:
a) at least one anionic surfactant,
b) at least one amphoteric and/or nonionic surfactant,
c) at least one cellulose-based cationic polymer,
d) at least one amino-functional silicone,
e) at least one organic acid, and
f) phenoxyisopropanol

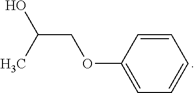

2. The hair treatment agent according to claim 1, comprising, based on its weight, about 0.5 to about 20% by weight of a) anionic surfactant(s).

3. The hair treatment agent according to claim 1, comprising, based on its weight, about 0.3 to about 10% by weight of amphoteric surfactant(s).

4. The hair treatment agent according to claim 1, comprising, based on its weight, about 0.3 to about 10% by weight of nonionic surfactant(s).

5. The hair treatment agent according to claim 1, comprising as cellulose-based cationic polymer(s) c), based on the weight of the agent, about 0.01 to about 3% by weight of at least one polymer chosen from the group of cationic cellulose polymers.

6. The hair treatment agent according to claim 1, comprising, based on its weight, about 0.01 to about 20% by weight of amino-functional silicone(s).

7. The hair treatment agent according to claim 1, comprising as d) amino-functional silicone(s) of the formula (Si-V) 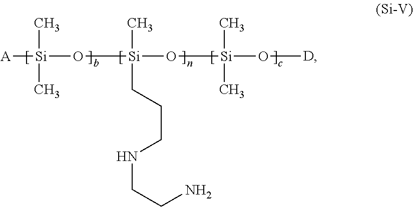
in which
A is a group chosen from —OH, —O—Si(CH₃)₃, —O—Si(CH₃)2OH, or —O—Si(CH₃)₂OCH₃,
is a group chosen from —H, —Si(CH₃)₃, —Si(CH₃)₂OH, or —Si(CH₃)₂OCH₃,
b, n and c are independently chosen from integers between 0 and about 1000,
provided that
i. n>0 and b+c>0; and
ii. at least one of the conditions A=—OH or D=—H is met.

8. The hair treatment agent according to claim 1, comprising, based on its weight, about 0.001 to about 10% by weight as e) organic acid(s) chosen from the group of formic acid, oxalic acid, maleic acid, lactic acid, tartaric acid, citric acid, or combinations thereof.

9. The hair treatment agent according to claim 1, comprising, based on its weight, about 0.01 to about 3% by weight of f) phenoxyisopropanol.

10. A method for treating hair, comprising applying a hair treatment to dry or damp hair, wherein the hair treatment agent comprises:
a) at least one anionic surfactant,
b) at least one amphoteric and/or nonionic surfactant,
c) at least one cellulose-based cationic polymer,
d) at least one amino-functional silicone,
e) at least one organic acid, and
f) phenoxyisopropanol 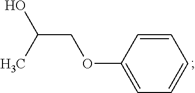
where the hair treatment agent is left on the hair for a time period of about 30 to about 300 seconds, and is rinsed out thereafter.

11. The hair treatment agent according to claim 1, comprising, based on its weight, about 0.01 to about 20% by weight of d) amino-functional silicone(s).

12. The hair treatment agent according to claim 1, comprising as d) amino-functional silicone(s) of the formula (Si-V) 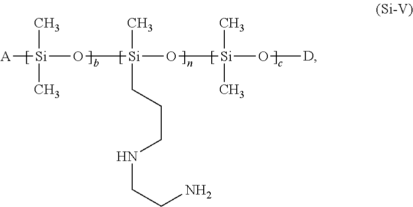
in which
A is —OH,
D is a group chosen from —H, —Si(CH₃)₃, —Si(CH₃)₂OH, or —Si(CH₃)₂OCH₃,
b, n and c are independently chosen from integers between 0 and about 1000,
provided that n>0 and b+c>0.

13. The hair treatment agent according to claim 1, comprising as d) amino-functional silicone(s) of the formula (Si-V) 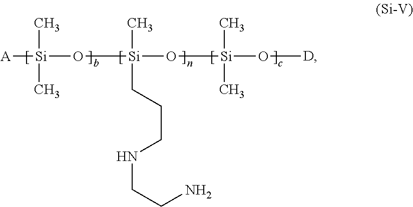
in which
A is a group chosen from —OH, —O—Si(CH₃)₃, —O—Si(CH₃)₂OH, or —O—Si(CH₃)₂OCH₃,
D is —H,
b, n and c are independently chosen from integers between 0 and about 1000,
provided that n>0 and b+c>0.

14. The hair treatment agent according to claim 1, comprising, based on its weight, about 0.001 to about 10% by weight of e) organic acid(s).

15. The hair treatment agent according to claim 1, comprising, based on the weight of the agent:
about 0.5 to about 20% by weight of a) anionic surfactant(s);
about 0.3 to about 10% by weight of b) amphoteric surfactant(s) or/or non-ionic surfactant(s);
about 0.01 to about 3% by weight of c) cellulose-based cationic polymer;
about 0.01 to about 20% by weight of d) amino-functional silicone(s);
about 0.001 to about 10% by weight of e) organic acid(s); and
about 0.01 to about 3% by weight of f) phenoxyisopropanol.

16. The hair treatment agent according to claim 15, comprising:
as d) amino-functional silicone(s) of the formula (Si-V) 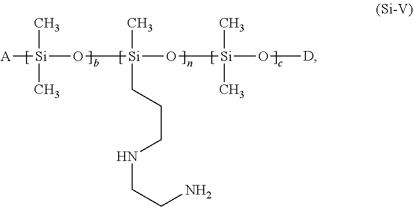
in which
A is a group chosen from —OH, —O—Si(CH₃)₃, —O—Si(CH₃)₂OH, or —O—Si(CH₃)₂OCH₃,
D is a group chosen from —H, —Si(CH₃)₃, —Si(CH₃)₂OH, or —Si(CH₃)₂OCH₃,
b, n and c are independently chosen from integers between 0 and about 1000,
provided that
i. n>0 and b+c>0; and
ii. at least one of the conditions A=—OH or D=—H is met; and
as e) organic acid(s) chosen from the group of formic acid, oxalic acid, maleic acid, lactic acid, tartaric acid, citric acid, or combinations thereof.
